(19) United States
(12) Patent Application Publication
(10) Pub. No.: US 2024/0167967 A1
(43) Pub. Date: May 23, 2024

(54) X-RAY IMAGING APPARATUS

(71) Applicants: Woorien Co., Ltd., Gyeonggi-do (KR); VATECH EWOO Holdings Co., Ltd., Gyeonggi-do (KR)

(72) Inventors: Taehee HAN, Gyeonggi-do (KR); In CHOI, Gyeonggi-do (KR)

(73) Assignees: Woorien Co., Ltd., Gyeonggi-do (KR); VATECH EWOO Holdings Co., Ltd., Gyeonggi-do (KR)

(21) Appl. No.: 18/512,598

(22) Filed: Nov. 17, 2023

(30) Foreign Application Priority Data
Nov. 17, 2022 (KR) 10-2022-0154511

(51) Int. Cl. G01N 23/046

(52) U.S. Cl. CPC G01N 23/046; G01N 2223/1016; G01N 2223/316; G01N 2223/3303

(57) ABSTRACT

An X-ray imaging apparatus is proposed. The proposed X-ray imaging apparatus is configured to include an imaging part including a generator part including the X-ray generator for emitting X-rays to an imaging target area and a sensor part including the X-ray sensor for receiving the X-rays transmitted through the imaging target area, a first driving part for rotating the generator part and sensor part about a rotation axis therebetween, and a second driving part for moving the X-ray sensor in a direction of rotation about the rotation axis with respect to the sensor part or a direction tangential to the rotation, wherein, during X-ray imaging, the first driving part rotates the imaging part reciprocally between first and second positions, and the second driving part moves the X-ray sensor either from the first position or from the second position.

### CROSS REFERENCE TO RELATED APPLICATION

The present application claims priority to Korean Patent Application No. 10-2022-0154511, filed Nov. 17, 2022, the entire contents of which are incorporated herein for all purposes by this reference.

### BACKGROUND OF THE INVENTION

### Field of the Invention

The present disclosure relates to an X-ray imaging apparatus and, more particularly, to an X-ray imaging apparatus capable of providing a CT image of an imaging target area relatively large compared to a width of a sensor.

### Description of the Related Art

X-ray CT images are secured by obtaining and reconstructing a plurality of frames of projection data of X-rays transmitted through an object to be imaged at various angles while rotating an X-ray generator and X-ray sensor in opposing directions with an examination object or part of the examination object to be imaged disposed therebetween. In this case, the object to be imaged is disposed within a field of view (FOV) of a CT imaging apparatus, and the CT imaging apparatus reconstructs a three-dimensional image of the FOV to provide a three-dimensional image of the object to be imaged. The X-ray CT imaging apparatus may provide a tomography image of a desired position and direction of a user along with a three-dimensional image of the object to be imaged, thereby being used in a variety of fields such as tumor diagnosis, dental care including implant surgery, and veterinary care.

In general, a large-area X-ray sensor is required for X-ray CT imaging, and as an X-ray sensor area increases, cost thereof significantly increases. In order to solve the resulting problem of expensiveness of X-ray CT imaging apparatus, the present applicant has proposed a technology that utilizes a small width X-ray sensor, but expands a FOV thereof by rotating the X-ray sensor during an imaging sequence and simultaneously moving the X-ray sensor separately in a tangential direction of a rotation trajectory.

However, according to the above technology, the amount of rotation of an X-ray generator and X-ray sensor required to perform one imaging sequence increases several times compared to a cone beam CT imaging sequence of a conventional method. New difficulties may arise in an apparatus configuration that requires expensive parts such as slip rings, and the like to solve a problem of connection wire twisting due to the increased rotation amount.

In addition, depending on an object to be imaged, there is also a case where rotating more than 360 degrees around a circumference of the object may not be available. For example, in a case where an object to be imaged is a head part including a human dental arch, an X-ray sensor may rotate more than once around a circumference of the head part. However, in a case where an object to be imaged is a head part including a dental arch of a quadrupedal animal, there is a problem that a rotatable range of the X-ray sensor is inevitably limited because the back of a neck is usually positioned behind a temporomandibular joint of the animal.

### Documents of Related Art

(Patent Document 1) Korean Patent No. 10-1740358 (May 22, 2017)

### SUMMARY OF THE INVENTION

An objective of the present disclosure is to provide an X-ray imaging apparatus capable of obtaining a CT image for an extended FOV compared to a width of the X-ray sensor by using an X-ray generator and X-ray sensor rotating in opposing directions in a rotation range limited to less than 360 degrees. According to the present disclosure in order to solve the above-mentioned problem, there is provided an X-ray imaging apparatus configured to include: an imaging part including a generator part including an X-ray generator for emitting X-rays to an imaging target area and a sensor part including an X-ray sensor for receiving the X-rays transmitted through the imaging target area; a first driving part for rotating the generator part and sensor part about a rotation axis therebetween; and a second driving part for moving the X-ray sensor in a direction of rotation about the rotation axis with respect to the sensor part or a direction tangential to the rotation, wherein, during X-ray imaging, the first driving part rotates the imaging part reciprocally between first and second positions, and the second driving part moves the X-ray sensor either from the first position or from the second position.

An angle between the first and second positions may be less than 360 degrees.

The generator part may further include a collimator for controlling the X-rays emitted from the X-ray generator to be directed toward the X-ray sensor.

The second driving part may move the X-ray sensor in a length less than or equal to a width of the X-ray sensor from at least one of the first and second positions.

The imaging part may be configured to emit and receive the X-rays while rotating in at least one direction.

The X-ray imaging apparatus according to the present disclosure may provide a CT image of the imaging target area as a result of receiving the X-rays through the X-ray sensor.

According to the present disclosure, an objective is to provide an X-ray imaging apparatus capable of obtaining a CT image for an extended FOV compared to a width of an X-ray sensor while limiting a counter-directional rotation range of an X-ray generator and the X-ray sensor to less than one rotation.

### BRIEF DESCRIPTION OF THE DRAWINGS

FIG. 1 a view schematically illustrating an X-ray imaging apparatus as seen from a side according to an exemplary embodiment of the present disclosure.

FIG. 2 a view schematically illustrating the X-ray imaging apparatus as seen from above according to the exemplary embodiment of FIG. 1.

FIG. 3 is a view illustrating a configuration of an imaging part in the X-ray imaging apparatus according to the exemplary embodiment in FIGS. 1 and 2.

FIGS. 4A to 4E are views schematically illustrating a CT imaging sequence of the X-ray imaging apparatus according to the exemplary embodiment of the present disclosure.

FIG. 5 is a view illustrating a FOV of a CT image according to the imaging sequence of FIGS. 4A to 4E.

### DETAILED DESCRIPTION OF THE INVENTION

Hereinafter, various exemplary embodiments of the present disclosure will be described with reference to the drawings. The technical idea of the present disclosure may be more clearly understood through the exemplary embodiments. The present disclosure is not limited to the exemplary embodiments described below. The same reference numerals indicate components of the same nature, and the description of components described with reference to the previous drawings may be omitted below.

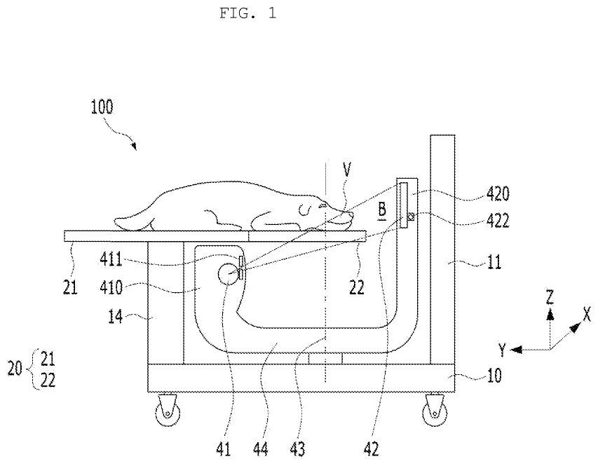

FIG. 1 a view schematically illustrating an X-ray imaging apparatus as seen from a side according to an exemplary embodiment of the present disclosure.

The present drawing illustrates a configuration of the X-ray imaging apparatus 100 according to the present exemplary embodiment in an easy-to-understand manner through a view in which an oral computed tomography (CT) image of a companion dog V as an example of an object to be imaged is being captured. The X-ray imaging apparatus 100 according to the present exemplary embodiment is configured to include: a cart-type frame 10; an imaging part 40 installed on the cart-type frame 10; a first driving part (not shown) for rotating the imaging part 40 relative to the cart-type frame 10; and a bed part 20 for supporting the object to be imaged. The imaging part 40 may be configured to include: a generator part 410; a sensor part 420; and a rotary arm 44 configured to connect the generator part 410 and the sensor part 420 to each other and be rotatable relative to a single rotation axis 43. Although not shown, the first driving part may be disposed inside the cart-type frame 10 or the rotary arm 44, so as to rotate the generator part 410 and the sensor part 420 about the rotation axis 43.

The generator part 410 is configured to include an X-ray generator 41 so as to emit cone beam-shaped X-rays B. The sensor part 420 is configured to include: an X-ray sensor 42 for receiving the X-rays B transmitted through an object to be imaged; and a second driving part 422 for linearly moving the X-ray sensor 42 in a width direction, i.e., in a direction tangential to a rotation trajectory of the sensor part 420, within the sensor part 420.

The bed part 20 extends from a position where the rotation axis 43 passes to an outside of a rotation radius of the generator part 410 so as to support the object to be imaged. The bed part 20 may be provided with a wide width part 21 having a relatively wide width and a narrow width part 22 having a relatively narrow width along a longitudinal direction from the outside. When the longitudinal direction of the bed part 20 is defined as a y-axis direction and a direction perpendicular to the y-axis on a horizontal plane parallel to a floor is defined as an x-axis direction, the rotation axis 43 may be configured to be movable in the x-axis direction. The horizontal movement of the rotation axis 43 is not limited to linear movement. However, it is advantageous for the rotation axis 43 to move in the x-axis direction in order to provide an imaging sequence suitable for a shape of a dental arch of a companion dog V which may be an object to be imaged and to avoid collision between the imaging part 40 and a front support part 11 or a rear support part 14, which is a vertical structure of the cart-type frame 10. However, there is no requirement for the rotation axis 43 to move while performing a CT imaging sequence. Referring back to the bed part 20, the wide width part 21 is disposed on the rear support part 14 so as to support a body weight of an animal to be imaged, and the narrow width part 22 is formed in a shape of a cantilever extending forward from the wide width part 21 and disposed to include a part intersecting the rotation axis 43.

In the above-described imaging part 40, the generator part 410 on which the X-ray generator 41 is installed has a lower height than the sensor part 420 on which the X-ray sensor 42 is installed. In the X-ray generator 41, X-rays B emitted through a collimator 411 diagonally penetrate an oral cavity part of the companion dog V to be imaged from bottom to top, and reach the X-ray sensor 42. Such a configuration of the bed part 20 and the imaging part 40 provides advantageous effects in several aspects. First, a rotation trajectory of the generator part 410 may pass under the bed part 20, which is advantageous in terms of apparatus miniaturization. In addition, emitting X-rays B from the X-ray generator 41 to the X-ray sensor 42 is enabled at an appropriate inclination, whereby it also helps improve the quality of CT images of oral cavities of animals having different sizes and shapes of the upper and lower jaws.

For reference, in the drawing, the bed part 20 is shown as installed on the rear support part 14 of the cart-type frame 10, but may also be configured with another device separated from the cart-type frame 10. Meanwhile, although not shown in the drawing, the narrow width part 22 of the bed part 20, on which a head part of an animal to be imaged is mainly positioned, may be further provided with side support parts (not shown) for supporting both sides of the head part, and the side support parts may be configured to enable adjustment or modification of positions.

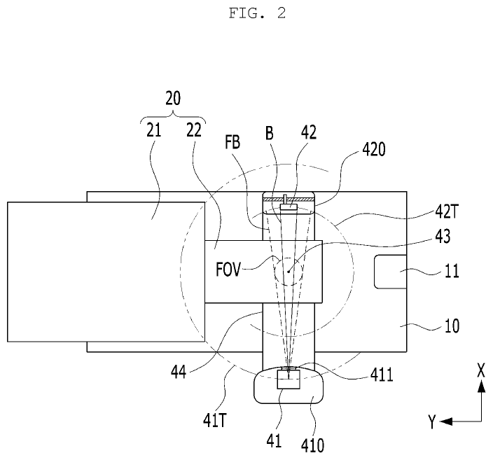

FIG. 2 a view schematically illustrating the X-ray imaging apparatus as seen from above according to the exemplary embodiment of FIG. 1.

The present drawing shows a state in which the imaging part 40 including the generator part 410, sensor part 420, and rotary arm 44 is rotated 90 degrees in a counterclockwise direction compared to that in FIG. 1. To facilitate understanding, the image of the animal V to be imaged shown in FIG. 1 is omitted. The present drawing may provide visual confirmation on a transmission path of X-rays B and a position of the X-ray sensor 42 when X-ray projection data of a unit frame is obtained, and a FOV relationship and the like on an assumption that the X-ray sensor 42 moves within the sensor part 420. The rotary arm is configured to rotate reciprocally around the rotary axis 43 in a predetermined angle range. Accordingly, the generator part 410 and the sensor part 420 move about the rotation axis 43 along arc-shaped trajectories 41T and 42T which are part of a circle. Hereinafter, for convenience of description, among reciprocal rotations, a clockwise rotation will be referred to as a first direction rotation, and a counterclockwise rotation will be referred to as a second direction rotation.

As shown above, the generator part 410 and the sensor part 420 are positioned on sides that are opposite from each other and centered around the rotation axis 43. When an imaging sequence is performed to obtain projection data required for obtaining a CT image for the FOV, the sensor part 420 proceeding from one side of the narrow width part 23 on which the head of the animal to be imaged is disposed to the other side via the front moves reciprocally in both directions along a sensor part movement trajectory 42T in a shape of an arc open toward the bed part 20. At this time, the generator part 410 moves reciprocally in both directions along a generator movement trajectory 41T proceeding to one side of the narrow width part 23 by passing under the wide width part 21 from the other side (i.e., the opposite side of the one side) of the narrow width part 23. The movement trajectory 42T of the sensor part 420 is positioned higher from the floor than the generator movement trajectory 41T.

FIG. 3 is a view illustrating a configuration of an imaging part in the X-ray imaging apparatus according to the exemplary embodiment in FIGS. 1 and 2.

As described above, the sensor part 420 is provided with the second driving part 422 for linearly moving the X-ray sensor 42 in the width direction within the sensor part 420, i.e., in the direction tangential to the rotation trajectory of the sensor part 420. The second driving part 422 is configured to cause positions of the X-ray sensor 42 to move in the width direction in steps when the sensor part 420 rotates reciprocally the arc-shaped movement trajectory 42T (see FIG. 2) several times and obtains X-ray projection data. That is, the CT imaging sequence proceeds involving step-by-step movement of the X-ray sensor 42.

Here, in an imaging sequence involving step-by-step movement of the X-ray sensor 42, the X-ray sensor 42 is maintained in a state of stopped relative movement at any one position of a first sensor position SP1 to an N-th sensor position SPN within the sensor part 420 when obtaining projection data while rotating in the first direction or second direction in performing the imaging sequence in which the imaging part 40 obtains the X-ray projection data by rotating reciprocally several times in order to obtain a CT image for the FOV. This refers to an imaging sequence performed in a method where the rotary arm 44 rotates from a first position A1 to a second position A2 or from the second position A2 to the first position A1 so that the sensor part 420 relatively moves the X-ray sensor 42 to another position among the first sensor position SP1 to the N-th sensor position SPN in a tangential direction of the movement trajectory and resumes obtaining of projection data along with rotation again after reaching one end of the movement trajectory 42T (see FIG. 2) . Through a configuration of such an imaging sequence, rather than the conventional CT imaging apparatus, the X-ray imaging apparatus according to the exemplary embodiment may provide an extended FOV compared to the width of the X-ray sensor 42.

Here, the first sensor position SP1 to the N-th sensor position SPN (where N is a natural number greater than 1, and N=5 in the present exemplary embodiment) may be disposed at intervals to correspond to an approximate width of the X-ray sensor 42 within the sensor part 420. It is preferable to ensure that there is no gap between a sensing area at a time when the sensor 42 is at one sensor position SPn and a sensing area at a time when the sensor 42 is at an adjacent sensor position SPn+1. In addition, a number N of positions that the X-ray sensor 42 has to pass through is determined according to a relationship between the width of the X-ray sensor 42 and a size of a CT image FOV to be provided. As the width of the X-ray sensor 42 becomes narrower and the size of the FOV becomes larger, the number N of the sensor positions also increases.

For example, the second driving part 422 may be configured to include a ball screw and a driving motor 422M therefor. The positions of the X-ray sensor 42 in the sensor part 420 may be sequentially changed, for example, in an order of the first sensor position SP1 to the N-th sensor position SPN or in the opposite order thereof. However, it is sufficient to go through a process of obtaining projection data involving rotation in the first direction or second direction by going through each of the first sensor position SPI to the N-th sensor position SPN at least once within the imaging sequence. There are no limitations on the orders of position movement.

In the X-ray imaging apparatus according to the present exemplary embodiment, the generator part 410 may be configured to include an X-ray generator 41 capable of emitting X-ray full beams corresponding to the entire width, which may be covered by the X-ray sensor 42, in the sensor part 420 through movement by the second driving part 422. However, the generator part 410 may include a collimator 411 by which an emitting range of X-rays B is limited to the width and position of the X-ray sensor 42 in order to prevent unrequired X-ray exposure to an object to be imaged. In this case, the collimator 411 maintains a width of the emitting range of X-rays B constant, but it is preferable to configure that an emitting direction (i.e., a relative direction with respect to an arbitrary reference position of the sensor part 420) is interoperative according to the step-by-step movement of the X-ray sensor. For example, the collimator 411 may be configured to include a shielding blade, a ball screw 412 for moving the shielding blade, and a driving motor 411M for the ball screw 412.

The driving motor 411M of the collimator 411, a driving motor 422M of the second driving part of the X-ray sensor 42, and an imaging part driving motor (not shown) for rotating the imaging part 40 about the rotation axis 43 may be controlled by a controller (not shown).

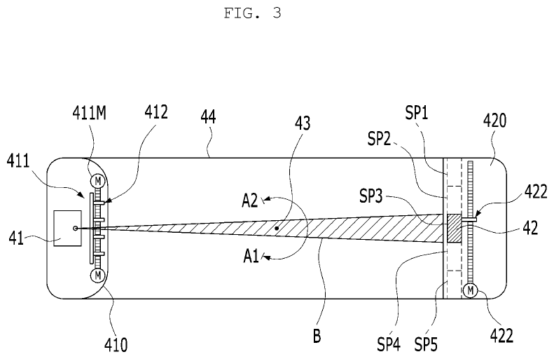

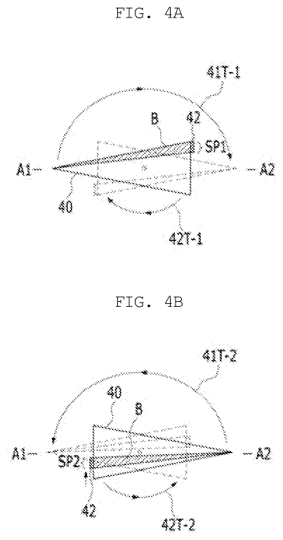

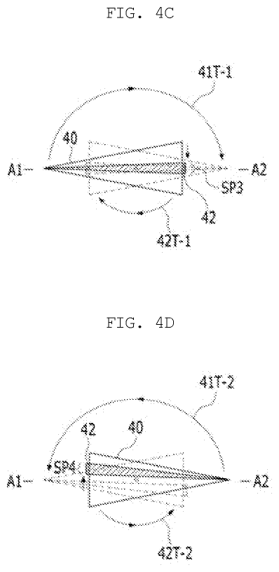

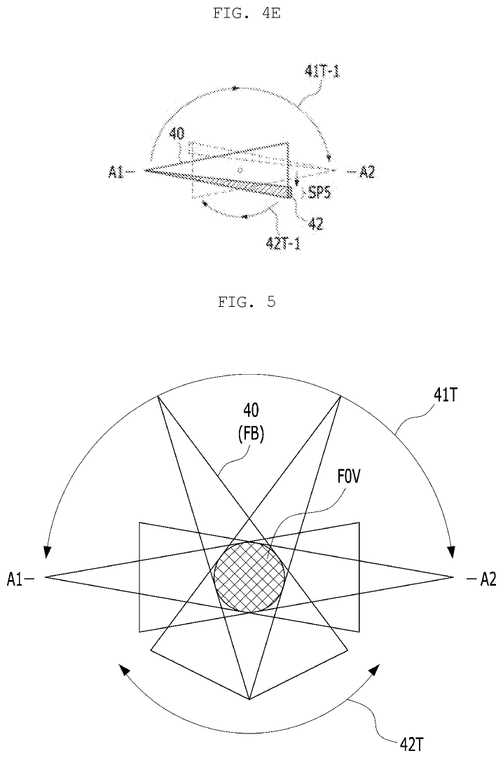

FIGS. 4A to 4E are views schematically illustrating a CT imaging sequence of the X-ray imaging apparatus according to the exemplary embodiment of the present disclosure.

According to the present exemplary embodiment, an imaging part 40 rotates reciprocally several times between a first position A1 and a second position A2, which have an angle difference of approximately 180 degrees from each other, and proceeds a CT imaging sequence for obtaining X-ray projection data. Hereinafter, the description will be made with reference to FIGS. 2 and 3 to aid understanding.

First, in a first step, as shown in FIG. 4A, in a state where an X-ray sensor 42 is positioned at a first sensor position SP1 within a sensor part 420, the imaging part 40 obtains projection data of a plurality of consecutive frames along with a section rotation until reaching from the first position A1 to the second position A2. In this process, a generator 41 moves along a movement trajectory 41T-1 in a first direction in its own reciprocal movement trajectory 41T, and the sensor part 420 also moves along a movement trajectory 42T-1 in a first direction in its own reciprocal movement trajectory 42T.

Next, in a second step, as shown in FIG. 4B, the X-ray sensor 42 is moved from the first sensor position SPI to a second sensor position SP2 within the sensor part 420. In that state, the imaging part 40 obtains projection data along with a section rotation until reaching back from the second position A2 to the first position A1. In this process, the generator 41 moves along a movement trajectory 41T-2 in a second direction in its own reciprocal movement trajectory 41T, and the sensor part 420 also moves along a movement trajectory 42T-2 in a second direction in its own reciprocal movement trajectory 42T.

Next, in a third step, as shown in FIG. 4C, the X-ray sensor 42 is moved from the second sensor position SP2 to a third sensor position SP3 within the sensor part 420. In that state, the imaging part 40 obtains projection data along with a section rotation until reaching back from the first position A1 to the second position A2. In this process, the generator 41 moves along the movement trajectory 41T-1 in the first direction in its own reciprocal movement trajectory 41T, and the sensor part 420 also moves along the movement trajectory 42T-1 in the first direction in its own reciprocal movement trajectory 42T.

Then after that, in a fourth step, as shown in FIG. 4D, the X-ray sensor 42 is moved from the third sensor position SP3 to a fourth sensor position SP4 within the sensor part 420. In that state, the imaging part 40 obtains projection data along with a section rotation in the second direction until reaching back from the first position A1 to the second position A2.

Last, in a fifth step, as shown in FIG. 4E, the X-ray sensor 42 is moved from the fourth sensor position SP4 to a fifth sensor position SP5 within the sensor part 420. In that state, the imaging part 40 obtains projection data along with a section rotation in the first direction until reaching back from the first position A1 to the second position A2.

The example of the imaging sequence described above corresponds to the exemplary embodiment in which the X-ray sensor 42 is configured to move five sensor positions in steps within the sensor part 420. In a case where a narrower width X-ray sensor is applied or even in a case where an X-ray sensor 42 passes through a greater number of sensor positions to obtain a larger FOV, an imaging sequence may be configured in a way of increasing the number of reciprocating rotations of the imaging part 40.

In addition, the above example of the imaging sequence is configured to obtain projection data not only when the imaging part 40 rotates in the first direction but also when the imaging part 40 rotates in the second direction, but may also be configured to obtain projection data only when the imaging part 40 rotates in any one of the first direction and second direction. In this case, for example, it may also be configured such that the X-ray sensor obtains projection data of a first step in a state of being positioned at the first sensor position while the imaging part rotates from the first position to the second position in the first direction, the X-ray sensor is moved from the first sensor position to the second sensor position when the imaging part returns from the second position to the first position, and then the X-ray sensor obtains projection data of a second step while the imaging part rotates again in the first direction.

FIG. 5 is a view illustrating a FOV of a CT image according to the imaging sequence of FIGS. 4A to 4E.

In the imaging part 40, the FOV of the CT image provided by the X-ray imaging apparatus according to the exemplary embodiment of the present disclosure is the same as the FOV formed by overlapping the entire sensor part width covered by the linear movement in the width direction of the X-ray sensor and the corresponding X-ray full beams. In other words, in the X-ray imaging apparatus according to the present disclosure, through the above-described imaging sequence, the imaging part 40 proceeds the scanning from the first position A1 to second position A2 (i.e., the obtaining of the projection data of consecutive frames) through a section rotation for each step in parallel with step-by-step movement of the X-ray sensor. The

X-ray projection data obtained by the X-ray sensor at the plurality of sensor positions may be reconstructed by an image processor, thereby providing a CT image. As a result, the CT image may be provided for an extended FOV compared to an actual width of the applied X-ray sensor.

The X-ray imaging apparatus having the above-described configuration may be applied to a CT imaging apparatus that primarily targets an animal as an object to be imaged. Typically, when performing CT imaging of the animal such as a companion dog and a companion cat, an imaging sequence is performed in a state where the target animal is anesthetized, so there is no problem even when the imaging part rotates reciprocally repeatedly. However, application targets of the present disclosure are not limited to the CT imaging apparatus for animals. Although there may be differences in suitability depending on body parts, the embodiment of the present disclosure may be applied to a CT imaging apparatus for human bodies as well, and naturally, may also be applied to an apparatus that targets other things as objects to be imaged.

What is claimed is:

1. An X-ray imaging apparatus comprising:
an imaging part comprising a generator part comprising an X-ray generator for emitting X-rays to an imaging target area and a sensor part comprising an X-ray sensor for receiving the X-rays transmitted through the imaging target area;
a first driving part for rotating the generator part and sensor part about a rotation axis therebetween; and
a second driving part for moving the X-ray sensor in a direction of rotation about the rotation axis with respect to the sensor part or a direction tangential to the rotation,
wherein, during X-ray imaging, the first driving part rotates the imaging part reciprocally between first and second positions, and the second driving part moves the X-ray sensor either from the first position or from the second position.

2. The X-ray imaging apparatus of claim 1, wherein an angle between the first and second positions is less than 360 degrees.

3. The X-ray imaging apparatus of claim 1, wherein the generator part further comprises a collimator for controlling the X-rays emitted from the X-ray generator to be directed toward the X-ray sensor.

4. The X-ray imaging apparatus of claim 1, wherein the second driving part moves the X-ray sensor in a length less than or equal to a width of the X-ray sensor from at least one of the first and second positions.

5. The X-ray imaging apparatus of claim 1, wherein the imaging part emits and receives the X-rays while rotating in at least one direction.

6. The X-ray imaging apparatus of claim 1, wherein a CT image of the imaging target area is provided as a result of receiving the X-rays through the X-ray sensor.
